(19) United States
(12) Patent Application Publication
(10) Pub. No.: US 2008/0248120 A1
(43) Pub. Date: Oct. 9, 2008

(54) Aerosol Composition and Method

(75) Inventors: James Anderson, Hull (GB); Andrea Duddington, Hull (GB); Wu Jin, Hull (GB); Simon Woolley, Hull (GB); Ivan Ye, GuanDong (CN)

Correspondence Address: NORRIS, MCLAUGHLIN & MARCUS, 875 THIRD AVE, 18TH FLOOR, NEW YORK, NY, 10022, US

(73) Assignee: RECKITT BENCKISER (UK) LIMITED, Slough, Berkshire (GB)

(21) Appl. No.: 12/091,989

(22) PCT Filed: Oct. 31, 2006
(86) PCT No.: PCT/GB2006/004067

§ 371 Date: May 30, 2008

(30) Foreign Application Priority Data
Nov. 1, 2005 (GB) 0522287.2

(51) Int. Cl. A61K 9/14; A61K 8/04; A61Q 15/00; A61Q 13/00

(52) U.S. Cl. 424/489; 512/4

(57) ABSTRACT

A method of dispensing periodic metered doses of a single phase aerosol composition wherein: the aerosol composition comprises a propellant and at least one active component selected from the group comprising fragrances, perfumes, air fresheners, deodorants and sanitisers; the metered dose spray rate is between 0.1 and 2 g/s of aerosol composition; and the mean particle size of each dose of the aerosol composition is between 1 μm and 40 μm.

The present invention describes a method of dispensing a single phase aerosol composition by use of a metered dose device, a composition for use in such a device, a method of manufacture of the composition and a device comprising the composition.

An aerosol is a common industry term to identify a large number of products which are dispensed as a mist, stream, spray, powder or even a foam. Pressurised cans are the typical vehicle for the formation of aerosols to dispense personal, household, industrial, and medical products, providing a low cost, easy to use method of dispensing such products. Typically, aerosol dispensers include a container, which contains a liquid product to be dispensed, such as soap, insecticide, paint, deodorant, disinfectant, air freshener, or the like. A pressurised propellant is used to provide a force sufficient to discharge the liquid product from the container. The user then actuates the aerosol dispenser by for example pressing an actuator button.

Optimal product delivery is only achieved by balancing the composition of the product in the pressurised container, the ratio of total propellant to total product and the delivery hardware (typically a valve and an actuator).

The propellant is a key component of the system. The two main types of propellants used in aerosol dispensers today are liquefied gas propellants, such as hydrocarbon, chlorofluorocarbon (CFC) and hydrofluorocarbon (HFC) propellants, and compressed gas propellants with a vapour pressure of greater than 101.3 kPa (14.7 psi), such as compressed carbon dioxide or nitrogen gas. The use of CFCs is, however, being phased out as they are heavily legislated against due to their potentially harmful effects on the environment through damage to the ozone layer. HFCs are not implicated.

In an aerosol dispenser using liquefied gas-type propellants, the container is loaded with the liquid product and propellant to a pressure approximately equal to, or slightly greater than, the vapor pressure of the propellant. Thus filled, the container still has a certain amount of space that is not occupied by liquid. This space is referred to as the “head space” of the dispenser assembly. Since the container is pressurized to approximately the vapor pressure of the propellant, some of the propellant is dissolved or emulsified in the liquid product. The remainder of the propellant is in the vapor phase and fills the head space. As the product is dispensed, the pressure in the container remains approximately constant as liquid propellant evaporates to replenish discharged vapour. Liquefied gas propellants keep the pressure constant in the aerosol can until the contents are exhausted, thus ensuring a consistent spray performance throughout the lifetime of the can. It is common to use a blend of propellant components to achieve best combination of solubility, economics, pressure and safety.

In contrast, compressed gas propellants (CO₂, N₂O, N₂) are not liquid in conventional aerosol containers; that is, they are present entirely in the vapor phase. The internal vapour pressure drops as the contents are depleted, causing changes in the rate and characteristics of the spray.

The propellant typically used to propel an air freshener liquid product from an aerosol dispenser is a liquefied gas propellant mixture of propane, normal butane and isobutane having a propellant pressure of the region of 40 psig at 70° F. (2.72 atm at 294K). “Propellant pressure” refers to the approximate vapor pressure of the propellant, as opposed to “can pressure,” which refers to the initial gauge pressure contained within a full aerosol container.

An air freshener requires that the propellant be present in the amount of at least approximately 29.5% by weight of the contents of the dispenser assembly in order to satisfactorily dispense the air freshener liquid product.

It has been observed that a reduction in the propellant content adversely affects the product performance. Specifically, reducing the propellant content in the aerosol air freshener resulted in excessive product remaining in the container after the propellant is depleted (product retention), an increase in the size of particles of the dispensed product (increased particle size, resulting in the particles “raining” or “falling out” of the air), and a reduction in spray rate, particularly as the container nears depletion. Reduction of the particle size can then only be achieved by reconfiguring the hardware of the dispenser, for example by incorporation of a “breakup bar” for inducing turbulence in a product/propellant mixture prior to the mixture being discharged from the spray head.

For a non-emulsion single phase system, the formulation requirements in order to be able to deliver a spray (coarse or fine) make it highly desirable that formulated product is homogeneous, i.e. active ingredients, solvent system, propellant under pressure should form a solution. This has been achieved thus far only by the use of very high levels of active ingredient (U.S. Pat. No. 5,935,554).

The property requirements of an aerosol dispenser—low fall out, minimal surface and component damage—as well as spray dryness or wetness, droplet size and rate of spray are determined by propellant concentration and vapour pressure as well as the solvent (if any) used in combination with the dispenser and valve hardware. An aerosol package therefore consists of many variables which are delicately balanced.

WO03/082477 discloses a system using a piezoelectric vibrating plate of atomising liquids which have minimal droplet size, wherein it is preferred that each droplet evaporates entirely before falling back onto an adjacent surface. Performance of such systems is said to be vitiated by a large droplet size as the droplet will not have time to fully evaporate before reaching the adjacent surface.

WO63/066115 further discloses a method and apparatus for evaporating multi-component liquids such as fragrances also using a piezoelectric vibrating plate wherein complete evaporation is assured by maintaining the size of the droplets, the liquid's component vapour pressures and the height through which the droplets fall according to a complex predetermined mathematical relationship.

US 2004/0223943 teaches that aerosols comprising hydrocarbon propellant are characterised by an overwhelming initial burst of scent which has short longevity in the air, as a result of producing a higher amount of small droplets. It is preferable therefore to use compressed gas propellant in order to be able to control the particle size and number of droplets. Preferred droplet sizes are between 20 and 60 microns.

U.S. Pat. No. 5,935,554 and U.S. Pat. No. 5,516,504 describe an aerosol spray dispenser comprising a metering device and a single phase composition for use therein which comprises high concentrations of active ingredient relative to the amount of propellant in order to minimise VOC release into the atmosphere. Only a 150 mg metering valve is disclosed, and no preferred droplet sizes or dispensing rates are disclosed.

WO02/072161 describes an apparatus which periodically dispenses an air freshening substance from a pressurised container into a room. No dispensing rates or droplet sizes are disclosed.

EP0897755 and EP1382399 describe a method for repelling and eliminating harmful organisms by intermittent spraying of a chemical liquid comprising a pesticide, either by piezoelectric or aerosol means, wherein the particle size distribution of the atomised particles is such that 90% by cumulative volume has a particle size of 20 μm or less. In the aerosol method, the diameter of the particles is adjusted by varying the volume ratio (in %) of the chemical liquid to the volume of the pressure vessel, such that in order to have 90% of particles having a size of ‘x’ μm, the volume ratio should also be ‘x’ %. No specific metering devices or valve sizes are disclosed.

When the fragrance is an oil, it often also contains a suitable auxiliary solvent, in a quantity of up to 25% w/w of the oil. These materials are used for e.g. solubilising or diluting solid and viscous perfume ingredients to improve handling and formulating, as well as optimising the overall vapour pressure of the formulation. The presence of such an auxiliary solvent may be useful to have a monophasic oil or to modulate surface tension of said oil. As example of suitable solvents, one may cite polar or non-polar low molecular weight solvent such as isoparaffins, paraffins, hydrocarbons, silicon oils, perfluorinated aliphatic ethers, glycol ethers, glycol ether esters, esters, or ketones. Non-restrictive examples of such solvents includes dimethicone or cyclomethicone, which are commercialized by Chemsil Silicon INC. under the trade names Cosmetic Fluid® 1288, and respectively Cosmetic Fluid® 1387, jojoba oil, perfluoroisobutyl methyl ether, diethyl phthalate, dipropylene glycol and isopropyl myristate. Ideal products of this type are ones which have little or no odour, a particularly preferred example being isopropyl myristate (IPM). It has been surprisingly been found that reducing the level of such auxiliary solvents significantly improves the performance of fragrance compositions.

It has surprisingly been found that by use of a metered dose aerosol spray device in combination with an appropriately selected aerosol opening, an aerosol composition comprising one or more active species can be delivered in nebulised form, that is to say as a mist of very fine particles. It has been found that the rate of delivery of the nebulised formulation is crucial, giving rise to superior sensorial performance for relatively low the concentrations of active ingredient in the formulation.

The extent of the nebulisation arises not only from the unique delivery system but also from a specific aerosol composition designed for the metered dosage method of delivery. This gives rise to minimal fall out and a satisfactory height of mist cloud when the device is activated and hence superior sensorial effect.

The increased sensorial effect is also believed to be due to the avoidance of habituation. This is as a result of the fragrance concentration in the air which is moving above and below the odour detection threshold between bursts, due to rapid dispersion.

An additional factor in the determination of the formulation is that it should be as cheap as possible.

From a safety perspective this combination of dosage method and formulation results in smaller dosages, therefore less propellant is being released into the air, and is therefore preferable to systems where higher amounts of flammable propellant are released.

It is in this context that the present invention describes a method for dispensing an aerosol composition in a metered dose system which possesses the performance properties required, namely:
good spray performance, i.e. low fall out
small droplet size
low regular dose
   without recourse to intricate and expensive techniques for particle formation such as piezoelectric or ultrasonic methods.

According to a first aspect of the invention, therefore, there is provided a method of dispensing periodic metered doses of a single phase aerosol composition wherein:
the aerosol composition comprises a propellant and at least one active component selected from the group comprising fragrances, perfumes, air fresheners, deodorants and sanitisers;
the metered dose spray rate is between 0.1 and 2 g/s of aerosol composition; and
the mean particle size of each dose of the aerosol composition is between 1 μm and 40 μm.

Preferably the single phase aerosol composition has an active concentration from between 0.1 and 20 wt %, preferably between 0.5 and 15 wt %, more preferably between 0.5 and 10 wt %, especially between 1 and 10 wt %. In a particularly preferred embodiment, the active concentration is between 8 and 8.5 wt %. Preferably the single phase aerosol composition has a viscosity of less than approximately 15 cP, preferably less than approximately 13 cP, preferably less than approximately 11 cP, preferably less than or equal to approximately 10 cP, especially 1.5-5 cP. Preferably the single phase aerosol composition has a surface tension of between 15 to 35 mN/m. Preferably the single phase aerosol composition has a vapour pressure of between 1 to 10 mPa. Preferably the single phase aerosol composition has a flash point of between 60 to 80° C. Preferably the dose amount per repeat dose expressed in mass units is between 2 and 20 mg. Preferably the dose amount per repeat dose expressed in volume units is between 2 and 25 mg.

Preferably the metered dose device has an exit hole of between 0.1 to 1.2 mm in diameter, preferably 0.2 to 11.0 mm, more preferably 0.2 to 0.8 mm, especially 0.25 to 0.75 mm. Preferably the metered dose device has a frequency of delivery (i.e. the time interval between repeating metered doses) of between 1 and 10 doses an hour, preferably between 2 and 8 doses per hour.

A preferred metered dose device comprises a solenoid operated device, especially a miniature solenoid valve as described in the following co-pending applications from the same applicant: GB 0427646.5, GB 0503098.6, GB 0503042.4, GB 0503095.2, GB 0521064.6, GB 0521061.2, GB 0521063.8 and GB 0521071.1, herein incorporated by reference.

According to a second aspect of the same invention, there is provided the use of a single phase aerosol composition, according to the method as hereinbefore described, comprising:
a. between 85 and 99.9 wt % of a propellant selected from the group comprising hydrocarbons, hydrofluorcarbons and dimethyl ether or a mixture thereof;
b. between 0.1 and 15 wt % of an active ingredient selected from the group comprising fragrances, perfumes, deodorants, air fresheners and sanitisers;
c. less than 1 wt % of an auxiliary solvent selected from the group comprising diethylene glycol, dipropylene glycol, triethyl citrate, isopropyl myristate and benzyl benzoate; and
d. from 0 to 10 wt % of a component other than the above;
   in a metered dose device, wherein a, b, c, d total 100.

Preferably in the composition as hereinbefore described the propellant (a) comprises butane. More preferably, propellant (a) is selected such that it satisfies pressure restrictions on aerosol can, particular examples being propellants which comprise butane 46, 70 or 30.

Preferred is a composition as hereinbefore described which comprises 90 to 99.5 wt % butane (a), more preferably 90 to 99 wt % butane (a), especially 90 to 95 wt % butane (a).

Preferred hydrofluorocarbons comprised by propellant (a) are HFC 152a and HFC134 or mixtures thereof.

Preferred is a composition as hereinbefore described which comprises 0.5 to 10 wt % of active ingredient (b), preferably 1 to 10 wt % of active ingredient (b), especially 5 to 10 wt % of active ingredient (b).

Preferred is a composition as hereinbefore described wherein active ingredient (b) is a fragrance or air freshener.

Preferably, the fragrance or air freshener is a fragrance comprising one or more volatile organic compounds which are available from perfumery suppliers such as Firmenich Inc., Takasago Inc., Noville Inc., Quest Co., International Flavors & Fragrances, and Givaudan-Roure Corp.

A wide variety of chemicals are known for perfumery, such as aldehydes, ketones, esters, alcohols, terpenes, and the like. Most conventional fragrance materials are volatile essential oils. A fragrance can be relatively simple in composition, or can be a complex mixture of natural and synthetic chemical components.

Natural fragrances include naturally derived oils such as oil of Bergamot, Bitter Orange, Lemon, Mandarin, Caraway, Cedar Leaf, Clove Leaf, Cedar Wood, Geranium, Lavender, Orange, Origanum, Petitgrain, White Cedar, Patchouli, Lavandin, Neroli, Rose absolute, and the like. Natural perfumes include the extracts of blossoms, stems and leaves, fruits, fruit peel, roots, woods, herbs and grasses, needles and branches, resins and balsams. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetivert oil, olibanum oil, galbanum oil, ladanum oil and lavendin oil.

Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams.

Synthetic types of fragrance compositions either alone or in combination with natural oils are described in U.S. Pat. Nos. 4,324,915; 4,411,829; and 4,434,306; incorporated herein by reference. Other artificial liquid fragrances include geraniol, geranyl acetate, eugenol, isoeugenol, linalool, linalyl acetate, phenethyl alcohol, methyl ethyl ketone, methylionone, isobornyl acetate, and the like.

It is, however, preferred to use mixtures of different perfume compounds which, together, produce an agreeable fragrance.

The following are also preferably used either individually or in the form of mixtures: dihydromyrcenol, lilial, lyral, citronellol, phenylethyl alcohol, α-hexylcinnamaldehyde, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, benzyl acetate, rose oxide, romillat, irotyl and floramat.

Preferably solvent (c) is diethylene glycol, dipropylene glycol or isopropylmyristate. In an especially preferred embodiment solvent (c) is isopropylmyristate.

The composition may also comprise up to 10 wt % of further adjuvants and/or excipients, such as but not restricted to corrosion inhibitors, preservatives, biocides, pH modifiers and buffers, surfactants, oil components, emulsifiers, stabilizers, polymers, silicone compounds, antioxidants, film formers, solubilizers, preservatives, dyes and the like.

In a particularly preferred embodiment of the second aspect of the invention as hereinbefore described there is provided a composition comprising:
between 90 and 95 wt % of butane 70 propellant;
between 5 and 10 wt % of a fragrances; and
less than 1 wt % of isopropylmistyrate;
   wherein all parts add up to 100.

In a further preferred embodiment of the first aspect of the invention as hereinbefore described there is provided a composition comprising:
between 94 and 99 wt % of HFC 152a propellant;
between 1 and 6 wt % of a fragrances; and
less than 1 wt % of isopropylmistyrate;
   wherein all parts add up to 100.

According to a third aspect of the invention, there is provided a method of manufacture of a composition as hereinbefore described in the second aspect which comprises the steps of:
combining the active ingredient with the remaining non-propellant component(s) (if present) to form a homogeneous mixture;
transferring the resultant mixture to an aerosol container;
sealing the container with a valve; and
pressuring the container and mixture with propellant (a).

According to a fourth aspect of the invention, there is provided an aerosol container comprising a composition as hereinbefore described.

Metal aerosol cans are usually made from tinplated steel, or aluminium. Tinplate aerosol cans are mostly made up of three components—a top containing the valve opening, a body and a bottom. Some two piece cans are also now available. Aluminium cans are usually made from single piece of aluminium metal. The tinplate used to make aerosol cans is low carbon mild steel sheet, coated with tin applied by electrodeposition. The thickness of the tinplate sheet used to make aerosol cans will vary, depending on the size of the can, the pressure specification, and whether it is for can bodies or end components. For can bodies the thickness will range from 0.18 mm to 0.25 mm, and for tops/bottoms they will be 0.28 mm to 0.43 mm. Unless otherwise specified, the layer of tin on the steel is the same thickness on both sides. Again the amount of tin will vary, ranging between 2.0 g/m² and 2.8 g/m². The internal surfaces of the tinplate will be either uncoated, or have a lacquer, or other material applied to give the metal better corrosion resistance. Tinplate combines the strength and formability of steel together with the corrosion resistance and good appearance of tin. Tin is a very soft metal and because the tin coating is very adherent it follows the movement of the steel base when tinplate is formed into the various components of an aerosol container.

Aerosol containers must be capable of withstanding the internal pressures generated during filling, and the subsequent transport, warehousing, and usage in the consumer's hands. They must also safely contain the product throughout the life of the aerosol. Aerosols are pressurised systems, and as such they are governed by legislation. This not only covers the manufacture of the empty can, but also its subsequent filling. Legislation governs the amount of product that may be filled into aerosol cans, and for safety reasons there will always be some space in the can which does not contain liquid, known as ‘head space’; because an aerosol is under pressure there must be sufficient space for the propellant to occupy, under all likely conditions. The amount of head space is greater when a compressed gas, such as air, is used, as these propellants operate at higher pressures than those for liquefied propellants.

Any standard form of commercial aerosol container may be employed to dispense compositions according to the first aspect of the present invention, such as but not restricted to aluminium or tin-plated steel containers, optionally comprising coatings and/or internal container linings, for example resin coatings such as epoxy resin resins. Capacities of the containers are typical for aerosol containers, preferably in the range 0.35 fl oz to 24 fl oz (10.3 ml to 706 ml), more preferably 0.35 fl oz to 1.02 fl oz.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention will now be further described by the embodiment described below, but is not restricted to the details thereof. The invention extends to any novel one, or any novel combination, of the features disclosed in this specification (including any accompanying claims, abstract and drawings), or to any novel one, or any novel combination, of the steps of any method or process so disclosed.

### EXAMPLES

### Example 1

8.5 wt % fragrance selected from the groups hereinbefore described was added to a standard 0.78 fl oz aluminium aerosol container of total volume 33 ml. The container was subsequently sealed with a standard continuous valve, and charged with 91.5 wt % Butane 70 propellant.

### Example 2

5.2 wt % fragrance selected from the groups hereinbefore described was added to a standard 0.78 fl oz aluminium aerosol container of total volume 33 ml. The container was subsequently sealed with a standard continuous valve, and charged with 94.8 wt % HFC-152a propellant.

In each case the resultant container was then incorporated into a solenoid operated metered dose aerosol spray device as described in the applicants' copending applications GB 0427646.5, GB 0503098.6, GB 0503042.4, GB 0503095.2, GB 0521064.6, GB 0521061.2, GB 0521063.8 and GB 0521071.1, herein incorporated by reference.

The resultant device gave excellent performance throughout the lifetime of the container, as evidenced by lack of “fall out”.

1. A method of dispensing periodic metered doses of a single phase aerosol composition wherein:
the aerosol composition comprises a propellant and at least one active component selected from the group comprising fragrances, perfumes, air fresheners, deodorants and sanitisers;
the method comprising the steps of:
dispensing the aerosol composition such that the metered dose spray rate is between 0.1 and 2 g/s of aerosol composition; and
the mean particle size of each dose of the aerosol composition is between 1 μm and 40 μm.

2. A method according to claim 1 wherein the single phase aerosol composition has an active component concentration from between 0.1 and 20 wt %.

3. A method according to claim 1, wherein the dose amount per periodic dose expressed in mass units is between 2 and 20 mg.

4. A method according to claim 1, wherein the dose amount per periodic dose expressed in volume units is between 2 and 25 μl.

5. A method according to claim 1, wherein the metered dose device has an exit hole of between 0.1 to 1.2 mm in diameter.

6. A method according to claim 1, wherein the metered dose device has a frequency of delivery of between 1 and 10 doses an hour.

7. A method according to claim 1, wherein the metered dose device comprises a solenoid operated valve.

8. The method according to claim 1 wherein the single phase aerosol composition comprises:
between 85 and 99.9 wt % of a propellant selected from the group comprising hydrocarbons, hydrofluorcarbons and dimethyl ether or a mixture thereof;
between 0.1 and 15 wt % of an active ingredient selected from the group comprising fragrances, perfumes, deodorants, air fresheners and sanitisers;
less than 1 wt % of an auxiliary solvent selected from the group comprising diethylene glycol, dipropylene glycol, triethyl citrate, isopropyl myristate and benzyl benzoate; and
from 0 to 10 wt % of a component other than the above;
in a metered dose device, wherein a, b, c and d total 100.

9. The method according to claim 8 wherein the propellant comprises butane.

10. The method according to claim 8 wherein the propellant comprises 90 to 99.5 wt % butane.

11. The method according to claim 8 wherein the propellant comprises hydrofluorocarbon HFC 152a or HFC 134 or a mixture thereof.

12. The method according to claim 8 wherein the single phase aerosol composition comprises 0.5 to 10 wt % of active ingredient (b).

13. The method according to claim 12 wherein active ingredient (b) is a fragrance or air freshener.

14. The method according to claim 8, wherein the single phase aerosol composition comprises an auxiliary solvent selected from: diethylene glycol, dipropylene glycol and isopropylmyristate.

15. The method according to claim 8, wherein the single phase aerosol composition comprises:
between 90 and 95 wt % of butane 70 propellant;
between 5 and 10 wt % of a fragrance(s); and
less than 1 wt % of isopropylmistyrate;
wherein all parts add up to 100.

16. The method according to claim 8, wherein the single phase aerosol composition comprises:
between 94 and 99 wt % of HFC 152a propellant;
between 1 and 6 wt % of a fragrance(s); and
less than 1 wt % of isopropylmistyrate;
wherein all parts add up to 100.

17. A method of manufacturing an aerosol container comprising a composition according to claim 8 which comprises the steps of:
combining active ingredient with the remaining non-propellant component(s) which may be present (if present) to form a homogeneous mixture;
transferring the resultant mixture to an aerosol container;
sealing the container with a valve; and
pressuring the container and mixture with the propellant (a).

18-20. (canceled)
